(19) United States
(12) Patent Application Publication
(10) Pub. No.: US 2013/0267742 A1
(43) Pub. Date: Oct. 10, 2013

(54) NITRILE CONTAINING HYDROCARBON FEEDSTOCK, PROCESS FOR MAKING THE SAME AND USE THEREOF

(71) Applicants: Delphine Minoux, Nivelles (BE); Cyril Revault, Le Havre (FR); Valentina De Grandi, Schaerbeek-Bruxelles (BE)

(72) Inventors: Delphine Minoux, Nivelles (BE); Cyril Revault, Le Havre (FR); Valentina De Grandi, Schaerbeek-Bruxelles (BE)

(73) Assignee: TOTAL RAFFINAGE MARKETING, Puteaux (FR)

(21) Appl. No.: 13/884,227

(22) PCT Filed: Dec. 27, 2011
(86) PCT No.: PCT/EP2011/074061

§ 371 Date: Jun. 6, 2013

(30) Foreign Application Priority Data
Dec. 28, 2010 (EP) 10306529.8

(51) Int. Cl. C07C 7/12; C07C 2/06

(52) U.S. Cl. CPC C07C 7/12; C07C 2/06
USPC 585/16; 585/251; 585/518; 585/822; 585/823

(57) ABSTRACT

The invention deals with a method for preparing a nitrogen-depleted hydrocarbon feedstock (315, 412, 514, 614, 714, 718, 814, 822) having (i) an initial boiling point comprised between 0° C. and +180° C. and a final boiling point comprised between 30° C. and 250° C., and (ii) an olefin content higher than 5 weight %, by contacting a hydrocarbon feedstock starting material (310, 313, 410, 411, 510, 512, 610, 612, 710, 712, 713, 810, 812, 813) with a clay sorbent material in a reaction vessel (33,43,53,63,73,73,83,83), wherein the nitrogen-depleted hydrocarbon feedstock (315, 412, 514, 614, 714, 718, 814, 822) has a total nitrogen/nitrile ratio (ppm/ppm) comprised between 1 and 5. Additional purification section allows improved units working time and lower maintenance.

The instant invention discloses a method for the preparation of specific nitrile containing hydrocarbon feedstock and subsequent conversion into higher boiling point cuts via olefin oligomerization and/or olefin alkylation onto aromatic moieties.

Refineries of today have to adapt to a continuously evolving and fluctuating market, requiring always more flexibility. It is especially the case with the gasoline/middle distillates markets, which have largely evolved during the years: a shift in product focus from gasoline to middle distillates is being observed in the current and future European market demands.

To respond to the above-mentioned disequilibrium, a nice way of readjusting the gasoline/diesel balance according to the market needs consists in upgrading at least part of the gasoline into middle distillates (jet, diesel).

In a typical refinery today, most of the C4-C8 molecules end up in the gasoline-pool. It is important to note that only around 5% of these molecules were initially present in the crude oil as delivered, while cracking during refinery processing creates the rest. About 50% of the C4's and 40% of the C5's that are produced during Fluidized Catalytic Cracking (FCC) are olefinic in nature. Currently the C4 olefins are used as feed for the alkylation and etherification units to create gasoline components with high octane and the higher olefins are generally directly blended into the gasoline pool.

In that context, a convenient solution that allows a renewed equilibrium between gasoline and distillates would be to convert unsaturated molecules (olefins and/or aromatics) contained in the gasoline feed into heavier molecules lying in the middle distillate range (i.e. diesel and kerosene) by selective oligomerization and/or alkylation of these unsaturated molecules.

The present invention relates to a process for the manufacture of higher molecular weight organic molecules from a stream of lower molecular weight molecules which contain contaminants brought in by the feedstock.

Oligomerization of olefinic streams is largely documented and is a widely used commercial process, but is subject to limitations.

Typically, oligomerization processes involve contacting lower olefins (typically mixtures of propylene and butenes) coming from Fluid Catalytic Cracker (FCC) and/or steam crackers with a solid acid catalyst, such as Solid Phosphoric acid (SPA) catalyst, crystalline molecular sieve, acidic ion exchange resin or amorphous acid material (silico-alumina).

With SPA catalyst, the pressure drop over the catalytic bed(s) increases gradually due to coking, swelling of the catalyst, and is therefore the limiting factor of a run duration, the unit being shutdown once the maximum allowable pressure drop has been reached.

With crystalline molecular sieve, acidic ion exchange resin or amorphous acidic material (silico-alumina), the limiting factor is usually no more the pressure drop along the catalytic bed but the reactor run length which is determined by the catalytic performances (shutdown when the catalytic activity has dropped to an unacceptably low level). The performances of such catalyst are therefore sensitive to poisons contained in the feedstock, which may considerably affect the cycle length.

Certain impurities such as sulfur containing contaminants and basic nitrogen have an adverse effect in the useful lifetime of the catalyst.

Among the sulfur containing contaminants, low molecular weight sulfur species are especially troublesome, as described in US 2008/0039669, i.e. aliphatic thiols, sulfides and disulfides. For example dimethyl-, diethyl-, and ethyl-methyl-sulphides, n-propane thiol, 1-butane thiol and 1,1-methylethyl thiol, ethyl-methyl- and dimethyl-disulphides, and tetrahydrothiophene.

Among the basic nitrogen contaminants, one can distinguish:
The strong organic Brønsted bases (characterized by at least one hydrogen atom bound to the nitrogen atom, and being proton acceptors), such as amines and amides, contribute to negatively affect the catalyst performances.
The other organic nitrogen compounds, called Lewis bases, have free electron pair on the nitrogen atom such as nitriles, morpholines or N-Methyl pyrrolidone. Though much weaker bases as compared to the Brønsted bases, they strongly deactivate the catalyst. The detrimental effect of such impurities has been discussed in US patent application publication 2008/0312484.

In some specific cases, the purity of the olefinic stream is not an issue:

It is the case when the stream involves very pure Fisher-Tropsch (FT) derived olefins (US2008/0257783 or WO2006/091986).

It is also the case in the fully integrated system MTG (Methanol-to-Gasoline) where olefinic streams are produced from the Methanol-To-Olefin process and oligomerized through the so-called MOGD process (Mobil Olefin to Gasoline and Distillate). The MOGD process, proposed by Mobil (U.S. Pat. No. 4,150,062; U.S. Pat. No. 4,227,992; U.S. Pat. No. 4,482,772; U.S. Pat. No. 4,506,106; U.S. Pat. No. 4,543,435) and developed between the seventies' and eighties', in fact used ZSM-5 zeolite as catalyst. The products obtained from the reaction of butenes are trimers and tetramers, characterized by a low branching degree. The gas oil fraction however is lower than that of the jet fuel fraction and consequently, even if this process offers good quality gas oil (cetane number >50), it is more interesting for the production of jet fuel than gas oil. In US2004/0254413 patent application, ExxonMobil pursued the Mobil development and introduced the new generation of MOGD. This invention uses two or more zeolite catalysts. Examples of zeolite catalysts include a first catalyst containing ZSM-5, and a second catalyst containing a 10-ring molecular sieve, including but not limited to, ZSM-22, ZSM-23, ZSM35, ZSM-48, and mixtures thereof. The ZSM-5 can be unmodified, phosphorous modified, steam modified having a micropore volume reduced to not less than 50% of that of the unsteamed ZSM-5, or various mixtures thereof.

ZSM-5 stands for Zeolite Sieve of Molecular porosity (or Zeolite Socony Mobil)-5, (structure type MFI-Mordenite Framework Inverted). ZSM-5 is an aluminosilicate zeolite mineral belonging to the pentasil family of zeolites. Its chemical formula is NaₙAlₙSi₉₆₋ₙO₁₉₂.16H₂O (0<n<27).

In a similar manner, Lurgi AG, Germany (WO2006/076942), has developed the Methanol to Synfuels (MTS) process, which is in principle similar to the MOGD process. The Lurgi route is a combination of simplified Lurgi MTP technology with COD technology from Sued Chemie (US5063187). This process produces gasoline (RON 80) and diesel (Cetane ˜55) in the ratio of approximately 1:4. Disclosed is a method for the production of synthetic fuels, wherein, in a first step, a gas mixture consisting of methanol and/or dimethyl ether and/or another oxygenate and water vapor is reacted at temperatures of 300-600° C. in order to form olefins with, preferably, 2-8 carbon atoms. In a second step, the olefin mixture thus obtained is oligomerized at an elevated pressure to form higher olefins with predominantly more than 5, preferably 10-20 carbon atoms. According to said method, a) the production of olefins in the first step is carried out in the presence of a gas flow which essentially consists of saturated hydrocarbons which are separated from the product flow of the second step and returned to the first step, and (b) the production of olefins is carried out in the second step in the presence of a flow of water vapor which is separated from the product flow of the first step and returned to the first step.

Above-discussed methods are hardly developable in the context of gasoline upgrading into distillates: commonly available olefinic feedstocks cause rapid deactivation of existing oligomerization catalysts, due to the presence of contaminants in the feed, which is a critical issue.

Catalytic cracking, usually fluid catalytic cracking (FCC), is a suitable source of cracked naphthas. Thermal cracking processes such as coking may also be used to produce usable feeds such as coker naphtha, pyrolysis gasoline, and other thermally cracked naphthas.

The process may be operated with a part of, or the entire gasoline fraction, obtained from a catalytic or thermal cracking step.

In case of alkylation reactions, the co-feed comprises a light fraction, boiling within the gasoline boiling range which is relatively rich in aromatics. A suitable refinery source for the light fraction is a reformate fraction. The reformate co-feeds usually contain very low amounts of sulfur as they have usually been subjected to desulfurization prior to reforming.

To cope with the contaminants issue, different techniques have been proposed:

A first technique consists in contacting the nitrogen and sulfur contaminated feedstock either with a hydrotreating catalyst at oxidized state (U.S. Pat. No. 6,884,916-Exxon) or with a metal oxide catalyst (U.S. Pat. No. 7,253,330) in the absence of hydrogen, ahead of the oligomerization section, thus limiting catalyst deactivation. The pretreatment is believed to convert small sulfur compounds into larger sulfur species, then into more sterically hindered molecules, no more entering the catalyst pores, and limiting catalyst deactivation.

Another convenient way (U.S. Pat. No. 7,186,874-Exxon) is to mitigate the adverse effect of the sulfur compounds on catalyst activity by appropriately adjusting the operating conditions of the process by, for example, temperature rising.

Removal of nitriles and other organic nitrogen-containing Lewis bases from the oligomerization feed may be achieved by a washing step with water (WO2007/104385-Exxon). Removal of basic nitrogen and sulfur-containing organic compounds by scrubbing with contaminant removal washes such as caustic, methyl-ethyl-amine (MEA), or other amines or aqueous washing liquids, is discussed in WO 2006/094010 (Exxon). This method allows contaminants to stand at acceptable levels (10-20 ppmwt S, trace levels for N) and therefore to limit catalyst deactivation prior to oligomerization and alkylation reactions.

Sorption techniques are also reported for nitrogen components removal from the feed. US2005/0137442 (UOP) discloses the use of molecular sieves catalysts (such as Y-zeolite) to remove the nitrogen-based contaminants present in an olefinic stream to be alkylated. Specificity of US2005/0137442 (UOP) lies in operating conditions: adsorption is conducted at a temperature of at least 120° C. to increase the nitriles adsorption capacity of the sorbent in the presence of water.

Purification section using molecular sieves is also reported in EP1433835 (IFP), where shaped MOR catalyst having a Si/AI atomic ratio of allows decreasing nitrogen content from 10 ppmwt to 0.2 ppmwt. US2008/0312484 (Exxon) shown that such a low nitrogen concentration can be tolerated in olefin-containing hydrocarbon feeds loaded in oligomerization sections.

WO2006/067305 (IFP) discloses a process for producing propylene from C4/C5 cut (from steam cracking or catalytic cracking). Prior to the steps of so-called “oligomerization/cracking”, the following purification sequence is used to remove contaminants: a selective hydrogenation is used to convert the dienes and acetylenic compounds into mono-olefins, then drying and desulfurization steps are performed by the use of different sorbents (3A, 13X molecular sieves).

Thus, state of the art processes do not use untreated refinery streams for oligomerization/alkylation but rather pure streams (such as ex-FT or ex-MTO olefins). As such, existing commercial solutions cannot give satisfactory results for untreated refinery streams to be valorized.

As explained above, gasoline pre-treatment represents a key step for life cycle improvement of oligomerisation catalysts. Indeed, such catalysts are very susceptible to poisoning by different species present in the feed, mostly dienes, sulfur compounds, oxygenated and nitrogenated compounds.

The evaluation of different sorbent materials in adsorption of nitrogen containing compounds in dynamic conditions under real total gasoline feed streams such as LCN (Light Cycle Naphtha) was considered.

Among available commercial sorbents, clays and derivatives were investigated with regards to their nitrogen retention capacity.

There are three or four main groups of natural clays: kaolinite, montmorillonite-smectite, illite, and chlorite.

U.S. Pat. No. 5,057,642 (Phillips Petroleum) claims a process for removing a basic impurity selected from the group consisting of ammonia, alkyl amines, cycloalkyl amines and aryl amines from a feed which comprises mono-olefins. Same U.S. Pat. No. 5,057,642 also states that removal of water from clay by thermal treatment is allegedly detrimental to preserve olefins in a feed: a minimal quantity of water left in the clay seems to be necessary to avoid competitive adsorption of monoolefins and/or to catalyze monoolefins dimerization. This phenomenon was also noticed in earlier Phillips Patent U.S. Pat. No. 4,351,980, where it was also observed that C2-C5 olefins were removed from a saturated hydrocarbon feed (mainly isobutane) containing low amount of olefins as impurities, by employing acid montmorillonite clays treated in slow nitrogen flow at 177° C. for 2.5 hours.

U.S. Pat. No. 5,057,642 carries out one experiment where the nitrogen containing flow is limited to 60 ppm added ammonia within an olefinic stream (see example, column 3, line 40).

U.S. Pat. No. 4,269,694 discloses the use of different sorbents, including optionally modified clays for impurities withdrawal from a feedstock. Impurities included silicon oils, used as antifoaming agents, and corrosion inhibitors such as amines.

Experiments conducted by the instant applicant shown that molecular sieves were efficient in total nitrogen removal from an olefin containing feedstock which contained nitrogen impurities of different types (alkyl amines, aryl amines, nitrogen inserted into aromatic rings (e.g. pyridines), cyanides (e.g. acetonitrile and heavier aliphatic nitriles such as butyronitrile). However, saturation started within ca. 30 hours on stream, under comparable operating conditions (see examples), which is considered poorly acceptable from industrial standpoint.

Other experiments conducted by the applicant using alumina adsorbent did not result in significant nitrogen retention.

On the contrary, the applicant found that the use of clays and especially acidic ones were less efficient in total nitrogen removal from an olefin containing feedstock (higher amount of total nitrogen after contacting the stream with clay), while saturation started within ca. 80 to 100 hours, which is more convenient for use in refining (using same experimental conditions as above).

Surprisingly, the applicant found that contacting a hydrocarbon feedstock having (i) an initial boiling point comprised between 0° C. and +180° C. and a final boiling point comprised between 30° C. and 250° C., and (ii) an olefin content higher than 5 weight %, with clay sorbent material resulted in (iii) a total nitrogen/nitrile ratio (ppm/ppm) comprised between 1 and 5.

Hence, a hydrocarbon feedstock having (i) an initial boiling point comprised between 0° C. and +180° C. and a final boiling point comprised between 30° C. and 250° C., (ii) an olefin content higher than 5 weight %, and (iii) a total nitrogen/nitrile ratio (ppm/ppm) comprised between 1 and 5 is produced at the outlet of a clay using section, when loading a reaction vessel with hydrocarbon feedstock having (i) an initial boiling point comprised between 0° C. and +180° C. and a final boiling point comprised between 30° C. and 250° C., (ii) an olefin content higher than 5 weight %, and (iii) a total nitrogen/nitrile ratio (ppm/ppm) higher than 5.

Accordingly, the invention relates to a method for preparing a nitrogen-depleted hydrocarbon feedstock having (i) an initial boiling point comprised between 0° C. and +180° C. and a final boiling point comprised between 30° C. and 250° C., and (ii) an olefin content higher than 5 weight %, by contacting a hydrocarbon feedstock starting material with a clay sorbent material in a reaction vessel, wherein the nitrogen-depleted hydrocarbon feedstock has a total nitrogen/nitrile ratio (ppm/ppm) comprised between 1 and 5.

The nitrogen-depleted hydrocarbon feedstock may be defined by the total nitrogen/nitrile ratio (ppm/ppm), which is advantageously comprised between 1 and 3, especially at the outlet of said clay sorbent material using section.

The hydrocarbon feedstock used as starting material is thus a feedstock comprising olefins and relatively high content of nitrogen compounds, preferably presenting a total nitrogen/nitrile ratio (ppm/ppm) higher than 1, preferably higher than 3, more preferably higher than 5, or especially between 6 and 10.

The solid materials useful as clay sorbent materials in this invention can be described as hydrated acid-treated smectite clays, such as montmorillonite, bentonite, vermiculite, hectorite, saponite, beidillinite and the like. In these clays, approximately every sixth aluminum ions has been replaced by a magnesium ion. This produces a crystal lattice with a negative charge which is neutralized by the absorption of metallic cations (such as Na+) on the surface. Their general chemical formula is (Na, Ca)_{0.3}(Al,Mg)₂Si₄O₁₀(OH)₂.nH₂O.

These surface cations are readily removed by treatment with an acid, such as HCl or H₂SO₄, wherein hydrogen ions are exchanged for the metallic ions. The acid-treated material can be designated as magnesium-substituted hydrogen montmorillonite. Sorbent materials of this type are sold commercially under the trade name of “Filtrol” by the Chemical Catalysts Group of Engelhard Corporation, Edison, N.J., and now BASF (further to takeover).

Specific acid-treated commercial clays designated as magnesium-substituted hydrogen montmorillonite include Filtrol Grade 71, Filtrol Grade F25, Filtrol Grade F24 and Tonsil CO—N, which are suitable clays for this purpose.

Preferably, the hydrocarbon feedstock starting material which is contacted with the clay has an olefin content higher than 20 weight %.

According to another aspect, the invention encompasses the use of a clay sorbent material for the preparation of a nitrogen-depleted hydrocarbon feedstock having (i) initial boiling point comprised between 0° C. and +180° C. and a final boiling point comprised between 30° C. and 250° C., (ii) an olefin content higher than 5 weight %, (iii) a total nitrogen/nitrile ratio (ppm/ppm) comprised between 1 and 5.

For this use, a hydrocarbon feedstock starting material is preferably presenting a total nitrogen/nitrile ratio (ppm/ppm) higher than 1, preferably higher than 3, more preferably higher than 5, or especially between 6 and 10 and includes olefins.

Preferably, used clay is selected among kaolinite, montmorillonite-smectite, illite and chlorite.

More preferably, said clay is hydrated acid treated smectite clay selected among montmorillonite, bentonite, vermiculite, hectorite, saponite, and beidillinite.

Particularly preferred the clay sorbent material is magnesium substituted hydrogen montmorillonite.

Clay sorbent materials used herein preferably have residual acidity greater than 3 mg KOH per gram of clay (acidity measured for example by acid-base proportioning).

Commercial clay sorbent materials acceptable for the preparation of a hydrocarbon feedstock according to above-mentioned specifications (i.e. (i) initial boiling point comprised between 0° C. and +180° C. and a final boiling point comprised between 30° C. and 250° C., (ii) an olefin content higher than 5 weight %, (iii) a total nitrogen/nitrile ratio (ppm/ppm) comprised between 1 and 5), can be selected among Filtrol F24, F124, F224, F25, F71 and Tonsil, such as Tonsil CO—N, as described above.

Most preferred is Filtrol F24 commercial clay.

According to another advantageous aspect, the invention includes a method for the preparation of the nitrogen-depleted hydrocarbon feedstock according to above-mentioned specifications (i.e. (i) initial boiling point comprised between 0° C. and +180° C. and a final boiling point comprised between 30° C. and 250° C., (ii) an olefin content higher than 5 weight %, (iii) a total nitrogen/nitrile ratio (ppm/ppm) comprised between 1 and 5, wherein the hydrocarbon feedstock starting material, preferably having a total nitrogen/nitrile ratio (ppm/ppm) higher than 1, preferably higher than 3, more preferably higher than 5, is contacted with the clay sorbent material, as defined above, within the reaction vessel, advantageously at a temperature comprised between the nitrogen-depleted hydrocarbon feedstock freezing point and final boiling point, with a liquid hourly space velocity (LHSV) lower than 4 h⁻¹, and a pressure comprised between 1 bar and 30 bar.

Preferably, the hydrocarbon feedstock starting material is contacted with said clay within said vessel at a temperature comprised between 0° C. and 100° C., with a LHSV between 3 h⁻¹ and 0.5 h⁻¹, and a pressure comprised between atmospheric pressure and 5 bar. In particular, the total nitrogen/nitrile ratio (ppm/ppm) in the starting material is comprised between 6 and 10.

Advantageously, the nitrogen-depleted hydrocarbon feedstock is further contacted with an adsorbent, wherein said adsorbent comprises one or more of molecular sieves, such as 13X and 3A, acidic ion-exchange resins, activated aluminas, spent FCC catalysts, MOF (Metal-Organic Framework), ASA (amorphous alumina-silica), NiMo, and catalysts guard beds, or mixture thereof, all known in the art and commercially available.

More advantageously, the adsorbent is selected among, or is a combination of one or more of 13X, 3A molecular sieves, ASA, NiMo, and MOF.

The applicant has found that contacting first a hydrocarbon feedstock starting material on a clay sorbent material, then within a molecular sieve, allowed high TOS (times of stream) without necessity for maintenance while preserving low nitrogen content of the resulting feedstock outflow.

When using dual nitrogen adsorption, preferred clay is F24, and preferred molecular sieve is 13X.

According to another aspect, the invention relates to a nitrogen-depleted hydrocarbon feedstock obtained by a method as described above. Specifically, this hydrocarbon feedstock exhibits (i) initial boiling point comprised between 0° C. and +180° C. and a final boiling point comprised between 30° C. and 250° C., (ii) an olefin content higher than 5 weight %, (iii) a total nitrogen/nitrile ratio (ppm/ppm) comprised between 1 and 5.

The total nitrogen/nitrile ratio (ppm/ppm) is advantageously comprised between 1 and 3, especially at the outlet of said clay sorbent material using section.

The instant invention also deals with the use of a nitrogen-depleted hydrocarbon feedstock as described above, within an olefin oligomerization and/or alkylation process.

The instant invention also deals with the use of a nitrogen-depleted hydrocarbon feedstock as described above, within an olefin oligomerization and/or alkylation unit.

In the present application, when the terms “between”, “higher” or “lower” are used, it should be understood that the corresponding interval may include or not the extreme value(s).

It has also been found an improved route to process untreated refinery streams, such as FCC or coker, into an oligomerization and/or alkylation reaction.

Accordingly, the invention also relates to a process for olefin oligomerization and/or alkylation of a nitrogen-depleted hydrocarbon feedstock containing olefins, comprising the successive steps consisting of:
(i) selective hydrogenation of a hydrocarbon feedstock starting material containing olefins,
(ii) treatment of the resulting hydrocarbon feedstock on a clay sorbent material to obtain at least one nitrogen depleted hydrocarbon feedstock, as described above,
(iii-a) oligomerization and/or (iii-b) alkylation of said at least one nitrogen depleted feedstock, for at least one middle distillate production.

In such a process, an olefin containing hydrocarbon feedstock, as starting material, successively undergoes (i) selective hydrogenation, (ii) treatment on a clay sorbent material to obtain at least one nitrogen depleted fraction, (iii) at least one middle distillate production (iii) via (iii-a) oligomerization of said nitrogen depleted fraction and/or (iii-b) alkylation of said at least one nitrogen depleted fraction.

The first step (i) consists in selective hydrogenation of di-olefins into mono-olefins within the hydrocarbon feedstock starting material containing olefins to avoid gum formation in downstream catalyst, allowing at the same time conversion of low molecular weight sulfur containing molecules (aliphatic thiols, sulfides or disulfides being especially troublesome) into heavier molecular weight sulfur containing molecules.

The step (i) is carried out using classical catalysts and usual operating conditions in the field of the invention, known to the one skilled in the art.

Preferably, the resulting stream is then fractionated in a splitter into a light cut (LCCS gasoline), and a heavier cut (HCCS or mixed MCCS/HCCS) (LCCS: Light Catalytic Cracked Stream, MCCS: Middle Catalytic Cracked Stream, HCCS: Heavy Catalytic Cracked Stream).

The further step (ii) consists in removing from the resulting hydrogenated hydrocarbon feedstock (or LCCS gasoline cut) the residual light N and S compounds by the use of a clay sorbent material, as described above, for obtaining the at least one nitrogen depleted hydrocarbon feedstock. The step (ii) may include a further step (ii-a) of contact of the said nitrogen-depleted feedstock with an adsorbent, wherein said adsorbent comprises one or more of molecular sieves, such as 13X and 3A, acidic ion-exchange resins, activated aluminas, spent FCC catalysts, MOF (Metal-Organic Framework), ASA, NiMo, and catalysts guard beds, all known in the art and commercially available, before entering the oligomerization/alkylation section.

Once purified in such a purification section, the nitrogen depleted hydrocarbon feedstock is then valorized into middle distillates by oligomerization and/or alkylation (step (iii)) as represented on FIGS. 3 to 8.

It has been found that oligomerization and/or alkylation catalyst lifetime is tremendously increased using this method. An hypothesis to explain improvement in catalyst lifetime in operation could be that the allegedly most detrimental sulfur containing compounds (S compounds) (such as aliphatic thiols, sulfides and disulfides: for example, dimethyl-, diethyl-, and ethyl-methyl-sulphides, n-propane thiol, 1-butane thiol and 1,1-methylethyl thiol, ethylmethyl- and dimethyl-disulphides) are converted within the selective hydrogenation unit by direct reaction of light sulfur containing molecules (e.g. here above-mentioned S compounds) with olefins or thiophenic compounds, thus leading to the formation of heavier S molecules, no more present in the LCCS cut issued from the splitter section.

Though thiophenic ring containing molecules are still present in the LCCS cut, they do not interact significantly with the acid sites of the oligomerization/alkylation catalyst.

The allegedly most detrimental nitrogen containing compounds (N molecules), if not converted into heavier N molecules in a similar manner as S molecules, are removed in the purification section (step (ii)) by adsorption on clay sorbent materials and optionally with an adsorbent. It is advantageous to use as adsorbents molecular sieves (3A, 13X, HY . . . ), acidic ion-exchange resins, activated aluminas such as SAS-351, MOF (Metal Organic Frameworks), amorphous alumina-silica (ASA), Spent FCC catalysts either alone or in combination.

Advantageously, the process includes a further step (iv) wherein at least one unreacted material is separated from said at least one middle distillate.

Advantageously, said at least one unreacted material is recycled at step (iii) for at least one middle distillate production.

In such a process, selectively hydrogenated feedstock (step (i)) may be splitted into at least two of LCCS (Light Catalytic Cracked Stream), MCCS (Middle Catalytic Cracked Stream), HCCS (Heavy Catalytic Cracked Stream) and fuel gas, prior to treatment of step (ii).

The alkylation of said at least one nitrogen depleted feedstock (step (iii)) is advantageously performed in presence of an aromatic containing hydrocarbon feedstock.

In a first embodiment of the above described process, the feedstock starting material successively undergoes (i) selective hydrogenation, (i-a) splitting into a light cut (LCCS) and a heavier cut (HCCS or mixed MCCS/HCCS), (ii) LCCS treatment on a clay sorbent material, optionally with a further treatment with an adsorbent, to obtain a nitrogen depleted LCCS, (iii) a first middle distillate production via (iii-a) oligomerization of said nitrogen depleted LCCS and/or (iii-b) alkylation of said nitrogen depleted LCCS.

Preferably, the alkylation step (iii-b) is performed in presence of an aromatic containing stream.

Advantageously, the process includes a further step (iv) wherein a first unreacted material is separated from said first middle distillate (step (iii)).

Advantageously, said first unreacted material is recycled at step (iii) for first middle distillate production.

The process may also include a further step (v) wherein said first middle distillate is further alkylated with a gasoline cut to produce a second middle distillate.

Preferably, the process may include a further step (vi) wherein a second unreacted material is separated from said second middle distillate and more particularly, said second unreacted material is recycled at step (v) for said second middle distillate generation.

In a second embodiment of the above described process, the feedstock starting material successively undergoes (i) selective hydrogenation, (i-a) splitting into a light cut (LCCS) and a heavier cut (HCCS or mixed MCCS/HCCS), (ii) said LCCS and said HCCS or mixed MCCS/HCCS undergo separate treatment on a clay sorbent material, optionally with a further treatment with an adsorbent, so as to remove nitrogen compounds, to obtain nitrogen depleted LCCS and nitrogen depleted HCCS or mixed MCCS/HCCS, (iii) a third middle distillate production via (iii-a) oligomerization and/or (iii-b) alkylation of said nitrogen depleted LCCS combined with said nitrogen depleted HCCS or mixed MCCS/HCCS.

A third unreacted material may be separated from said third middle distillate. A gasoline cut may also be separated from said third middle distillate. Preferably, said third unreacted material is recycled at step (iii) for said third middle distillate generation.

In a third embodiment of the above described process, the feedstock starting material successively undergoes (i) selective hydrogenation, (i-a) splitting into a light cut (LCCS) and a heavier cut (HCCS or mixed MCCS/HCCS), (ii) said LCCS and said HCCS or mixed MCCS/HCCS undergo separate treatment on a clay sorbent material, optionally with a further treatment with an adsorbent, so as to remove nitrogen compounds, to obtain nitrogen depleted LCCS and nitrogen depleted HCCS or mixed MCCS/HCCS, (iii-a) a fourth middle distillate production through oligomerization of said nitrogen depleted LCCS, (iii-b) a fifth middle distillate generation through alkylation of said nitrogen depleted HCCS or mixed MCCS/HCCS.

Preferably, alkylation is performed in presence of an aromatic containing stream.

Unreacted olefins are advantageously separated from said fourth middle distillate. For example, said unreacted olefins are recycled at step (iii-a) for fourth middle distillate production.

Fourth middle distillate may also be alkylated with nitrogen depleted HCCS or mixed MCCS/HCCS and said aromatic containing stream at step (iii-b) to produce said fifth middle distillate. Advantageously, unreacted material is then separated from said fifth middle distillate, and preferably, said unreacted material is recycled at step (iii-b).

In any of the embodiments of the above described process, said adsorbent comprises one or more of molecular sieves, acidic ion-exchange resins, activated aluminas, spent FCC catalysts, MOF (Metal-Organic Framework), ASA, NiMo, and catalysts guard beds. Besides, the process is classically implemented using appropriate reaction vessels or units, classically used in the field of the invention.

Preferably, the adsorbent is selected among, or is a combination of one or more of 13X, 3A molecular sieves, ASA, NiMo, and MOF.

Advantageously, the sorbent clay material and optionally the adsorbent used in any above described method is loaded into a purification section located in a guard bed capacity.

With regards to purification section (step (ii)) a guard bed reactor may be operated on a swing cycle with two beds, one bed being used on stream for contaminant removal and the other on regeneration in the conventional manner. If desired, a three-bed guard bed system may be used on a swing cycle with the two beds used in series for contaminants removal and the third bed in regeneration. With the three-bed guard bed system used to achieve low contaminant levels by the two-stage series sorption, the beds will pass sequentially through a three-step cycle of regeneration. A three-bed guard bed system allows better use of guard bed sorption capacity since non-used sorbent sent in regeneration is lowered, if not eliminated. Typically, a three-bed guard bed system may be operated as follows: Step 1: feedstock flows into first then second guard bed, third one being isolated and under regeneration. Step 2: once first guard bed is saturated in impurities, the latter is isolated and regenerated, and feedstock now flows into second then third guard beds. Step 3: once second guard bed is saturated in impurities, the latter is isolated and regenerated, and feedstock now flows into third then first guard beds. Step 4: go to step 1.

A plural reactor system may be employed with inter-reactor cooling for oligomerization and/or alkylation, whereby exothermal reaction can be carefully controlled to prevent excessive temperature above the normal moderate range.

The oligomerization and/or alkylation reactor can be of isothermal or adiabatic fixed bed type or a series of such reactors or a moving bed reactor. The oligomerization/alkylation may be performed continuously in a fixed bed reactor configuration using a series of parallel “swing” reactors. Herein used catalysts have been found to be stable enough. This enables the oligomerization/alkylation process to be performed continuously in two parallel “swing” reactors wherein, when one or two reactors are in operation, the other reactor is undergoing catalyst regeneration. Catalysts used in the method may be regenerated. Regeneration may be done several times.

An object of the above described process is to convert olefins containing stream into heavier hydrocarbons enriched distillate, employing a continuous multi-stage catalytic technique. A plural reactor system may be employed with inter-reactor cooling, whereby the exothermal reaction can be carefully controlled to prevent excessive temperature above the normal moderate range. Preferably, the maximum temperature differential across only one reactor does not exceed 75° C. Optionally, the pressure differential between the two stages can be utilized in an intermediate flashing separation step.

The following types of acid catalysts can be used in oligomerization and/or alkylation:

Amorphous or crystalline alumosilicate or silicaalumophosphate in H-form, optionally containing alkali, alkali-earth, transition or rare-earth elements, selected from the group:

MFI (e.g. ZSM-5, silicalite-1, boralite C, TS-1), MEL (Si/Al >25) (e.g. ZSM-11, silicalite-2, boralite D, TS-2, SSZ-46), ASA (amorphous silica-alumina), MSA (mesoporous silica-alumina), FER (e.g. Ferrierite, FU-9, ZSM-35), MTT (e.g. ZSM-23), MWW (e.g. MCM-22, PSH-3, ITQ-1, MCM-49), TON (e.g. ZSM-22, Theta-1, NU-10), EUO (e.g. ZSM-50, EU-1), ZSM-48, MFS (e.g. ZSM-57), MTW, MAZ, SAPO-11, SAPO-5, FAU (e.g. USY), LTL, BETA, MOR, SAPO-40, SAPO-37, SAPO-41, MCM-41, MCM-48 and a family of microporous materials consisting of silicon, aluminum, oxygen and optionally boron, Al₂O₃, and mixtures thereof.

Amorphous alumosilicates or silicaalumophosphates in H-form optionally modified by addition of halogens (Fluorine preferred) such as MSA (mesoporous silica-alumina) can also be used.

Above-mentioned catalysts can be subjected to an additional treatment before use, including ion exchange, modification with metals such as alkali, alkali-earth and rare earth metals, steaming, treatment in an alkaline medium, acid treatment or other dealumination methods, phosphatation, surface passivation by silica deposition or combination thereof.

The amount of alkali, alkali-earth, transition or rare-earth elements is in the range 0.05-10 wt %, preferably from 0.1 to 5 wt %, more preferably from 0.2 to 3 wt % (wt % stands for weight percent).

Preferred alkali, alkali-earth or rare-earth elements are selected among Na, K, Mg, Ca, Ba, Sr, La, Ce, and mixtures thereof.

Above-mentioned catalysts may be additionally doped with further metals. In this respect, and according to another embodiment of the invention, Me-catalysts (Me=metal) containing at least 0.1 wt % are used. Preferably, the metal is selected from the group of Zn, Mn, Co, Ni, Ga, Fe, Ti, Zr, Ge, Sn, Cr, and mixtures thereof.

Those atoms can be inserted into the tetrahedral framework through a [MeO₂] tetrahedral unit. Incorporation of the metal component is typically accomplished during synthesis of the molecular sieve. However, post-synthesis ion exchange or impregnation can also be used. In post-synthesis exchange, the metal component will be introduced as a cation on ion-exchange positions at an open surface of the molecular sieve, but not into the framework itself.

The selected materials could be subjected to a different treatment before use in the reaction, including introduction of phosphorous, ion exchange, modification with alkali, alkali-earth or rare earth metals, steaming, acid treatment or other dealumination methods, surface passivation by silica deposition or combination thereof.

The catalyst can be a blend of materials as depicted above, and/or can be further combined with other materials that provide additional hardness or catalytic activity to the finished catalyst product (binder, matrix).

The method described above permits to treat a feedstock issued from FCC, coker, flexi-coker, visbreaker, steam cracker, hydrocracker, for example from DHC (distillate hydrocracker) or MHC (mild hydrocracker) hydrocracker, preferably from FCC or coker.

The final boiling point of the feedstock may be below 200° C., preferably below 165° C.

The initial boiling point of the feedstock may be above −50° C., preferably above 0° C., more preferably above +25° C.

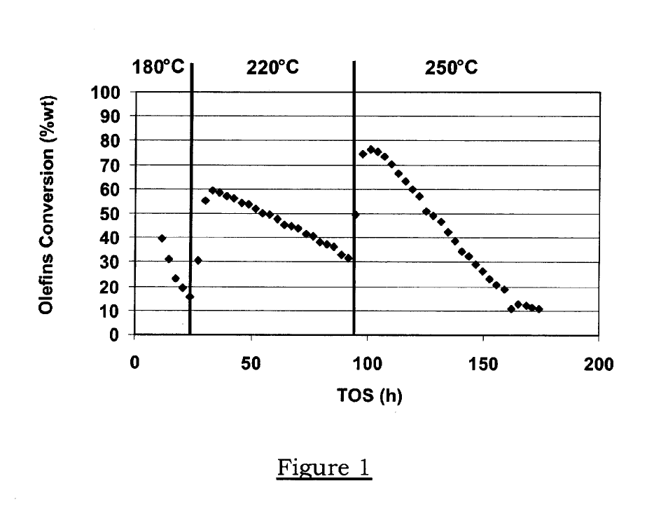

The invention is now described with reference to appended FIGS. 1-13, which depict different non-limitative methods for the preparation of middle distillate cuts starting from olefin containing lighter cuts, e.g. ex-FCC, ex-coker or ex-DHC gasoline cuts.

FIG. 1 is a graph showing olefin conversion (% wt) as a function of TOS (Time On Stream) when no purification section is used. (See example 1 for details)

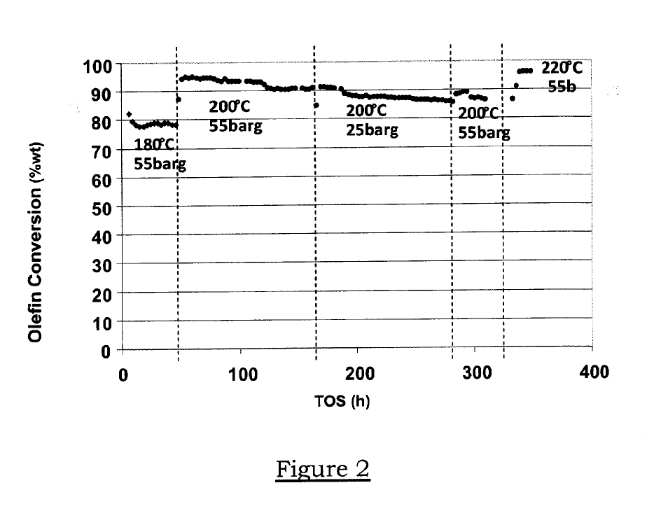

FIG. 2 shows olefin conversion rate (% wt) as a function of TOS when a purification section is used. (See example 2 for details)

FIG. 3 represents an oligomerization method using a purification section and optionally a recycle of unreacted stream.

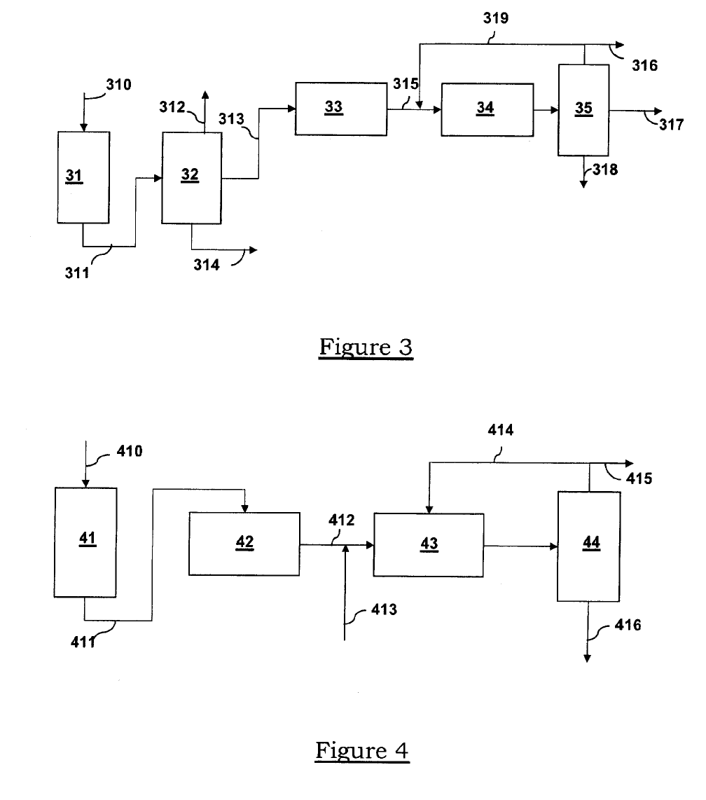

FIG. 4 shows an oligomerization and alkylation process scheme using a purification section and optionally a recycle of unreacted stream.

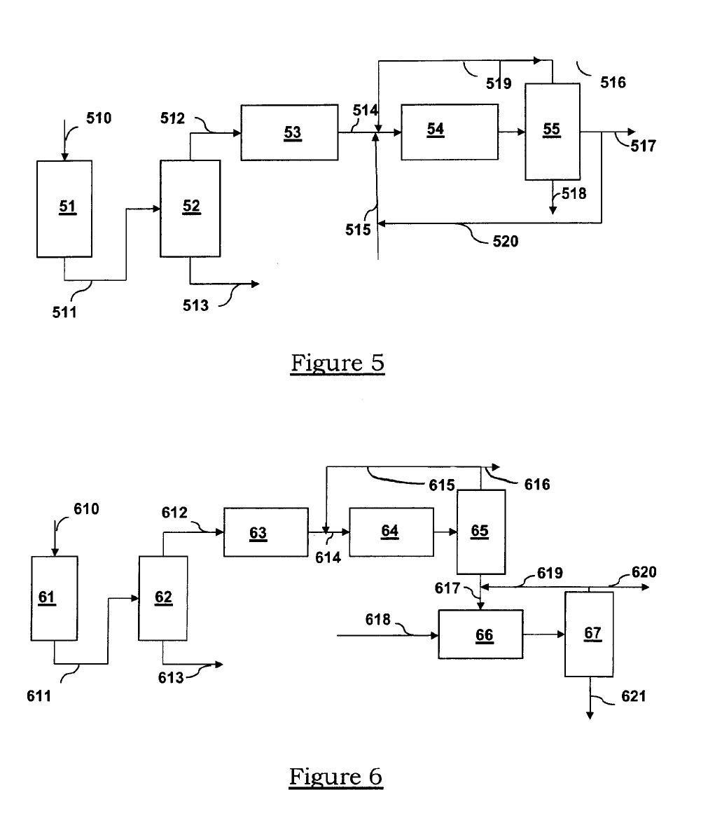

FIG. 5 illustrates another alkylation method using a purification section and optionally a recycle of unreacted olefin and aromatic streams.

FIG. 6 represents another oligomerization and alkylation process using a purification section and optionally a recycle of unreacted olefin and aromatic streams.

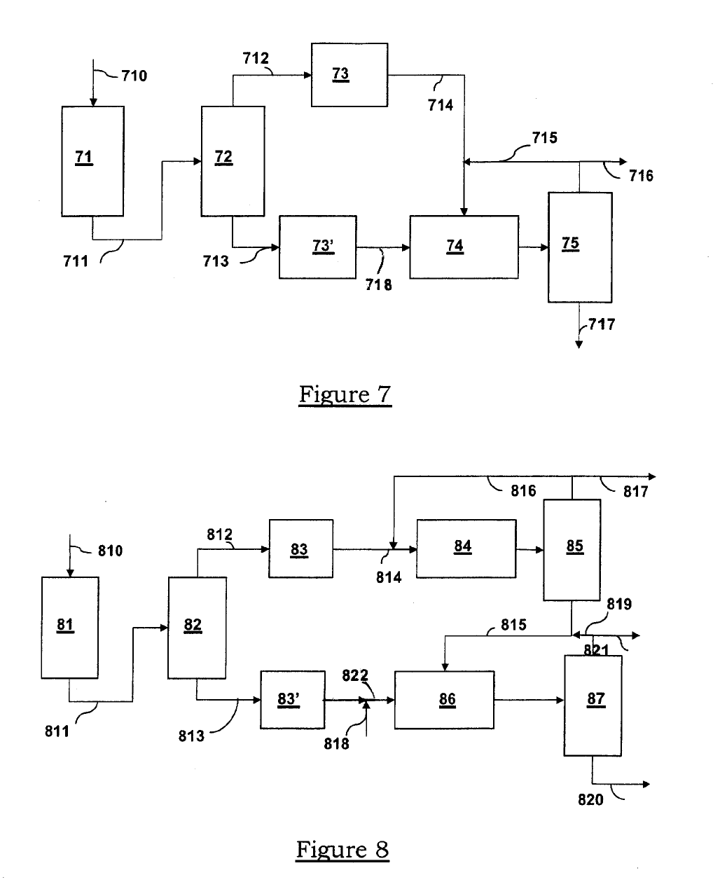

FIG. 7 shows a one-pot oligomerization and alkylation scheme using a purification section and optionally a recycle of unreacted olefin stream.

FIG. 8 represents an oligomerization and alkylation scheme of full olefin containing refinery stream using a purification section and optionally a recycle of unreacted olefin and aromatic streams.

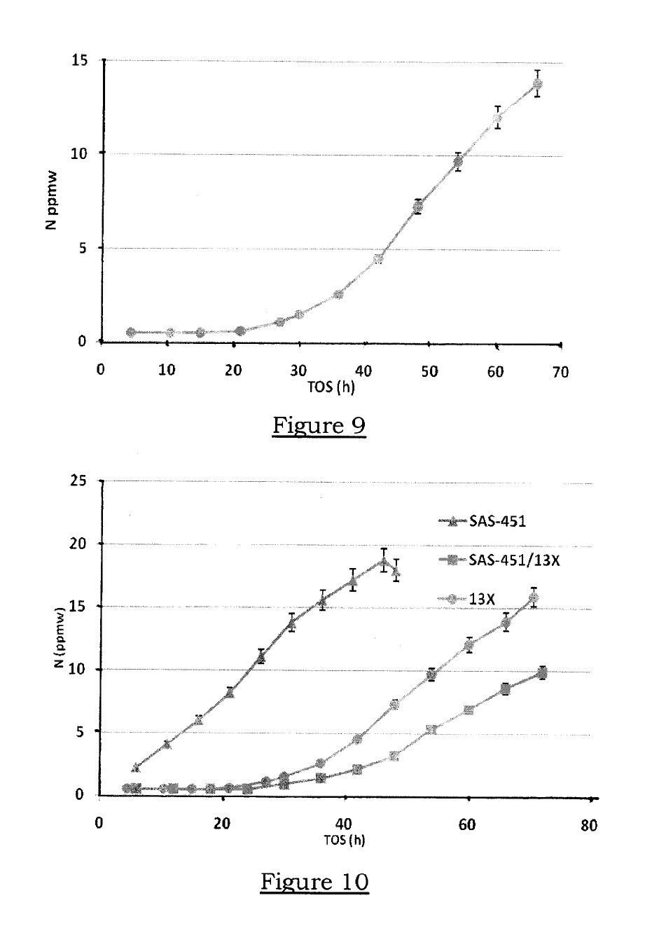

FIG. 9 presents the variation of total nitrogen content of a second LCN stream at the outlet of a reactor containing 13X molecular sieves (in part per million of nitrogen weight) as a function of TOS (Time On Stream).

FIG. 10 presents the variation of total nitrogen content of second LCN stream at the outlet of a reactor containing 13X molecular sieves or activated alumina SAS-451, or their combination (in part per million of nitrogen weight) as a function of TOS (Time On Stream).

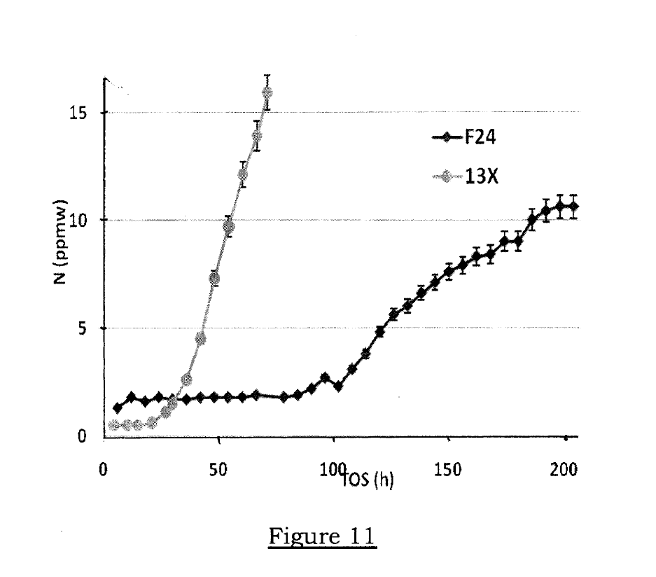

FIG. 11 presents the variation of total nitrogen content of second LCN stream at the outlet of a reactor containing 13X molecular sieves or Filtrol clay F24, each alone (in part per million of nitrogen weight) as a function of TOS (Time On Stream).

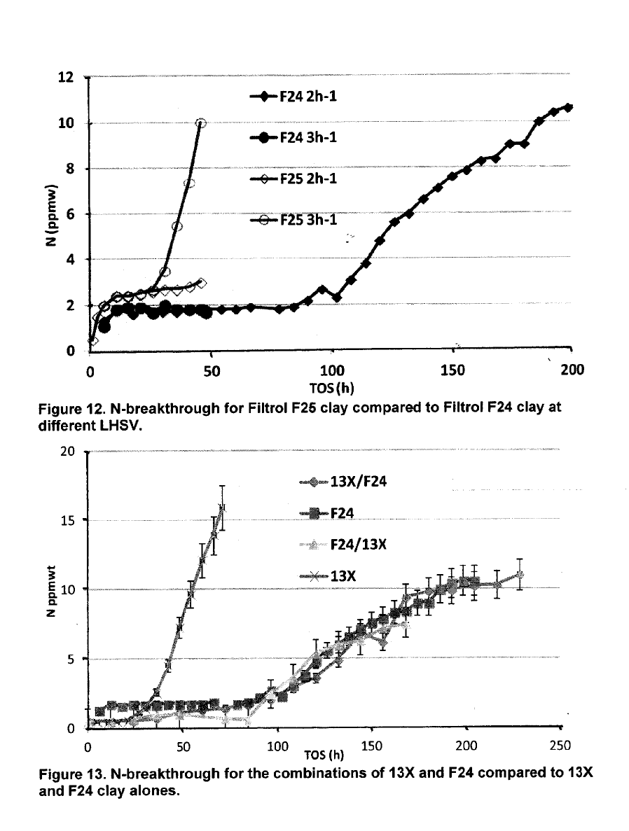

FIG. 12 displays the N-breakthrough for Filtrol F25 clay compared to Filtrol F24 clay at different LHSV.

FIG. 13 presents the N-breakthrough for the combinations of 13X and F24 compared to 13X and F24 clays alone.

FIG. 3 represents an oligomerization method using a purification section and optionally a recycle of unreacted stream.

The untreated refinery stream 310 is fed to a Selective Hydrogenation Unit (SHU) 31. The dedienized refinery stream 311 thus obtained is fed to a splitter 32 wherein it is separated in fuel gas 312, LCCS 313 (FBP=60° C. to 100° C.) and HCCS 314 (Boiling point cut ranges from 60-170° C. to 100-170° C.).

In the present application, FBP stands for Final Boiling Point and IBP stands for Initial Boiling Point.

LCCS 313 is then fed to a purification unit 33 for removal of nitrogen by adsorption on clay sorbent material, optionally further with an adsorbent as previously defined.

The purified LCCS stream 315 obtained is fed to an oligomerization unit 34, the obtained products being separated in a further splitter 35 into a gasoline 316, aromatics 317 and middle distillate 318.

The part of the gasoline 316, consisting of unreacted olefins, may optionally be recycled back into the oligomerization unit 34 via the line 319.

FIG. 4 shows an oligomerization and alkylation process scheme using a purification section and optionally a recycle of unreacted stream.

The untreated refinery stream 410 (for example LCN (Light Cracked Naphtha)) is fed to a Selective Hydrogenation Unit, SHU 41. The dedienized refinery stream 411 thus obtained is purified in a purification units 42 for removal of nitrogen by adsorption on clay sorbent material, optionally further with an adsorbent as previously defined.

The purified stream 412 is fed to an oligomerization unit 43. Optionally, an aromatic stream 413 may be co-fed with stream 412.

The effluents issued from the oligomerization unit 43 are separated in a splitter 44 into gasoline 415 and middle distillates 416.

The part of the gasoline 415, consisting of unreacted material, may optionally be recycled back into the oligomerization unit 43 via the line 414.

FIG. 5 shows an alkylation process scheme using a purification section and a recycle of unreacted olefin and aromatic streams.

The untreated refinery stream 510 is fed to a Selective Hydrogenation Unit (SHU) 51. The dedienized refinery stream 511 thus obtained is fed to a first splitter 52 wherein it is separated in LCCS 512 (FBP=60° C. to 100° C.) and M/HCCS 513 (Boiling point cut ranges from 60-170° C. to 100-170° C.).

LCCS 512 is then fed to a purification unit 53 for removal of nitrogen by adsorption on clay sorbent material, optionally further with an adsorbent as previously defined.

The purified LCCS stream 514 obtained is fed to an alkylation unit 54, the obtained products being separated in a further splitter 55 into a gasoline 516, aromatics 517 and middle distillates 518.

The part of the gasoline 516, consisting of unreacted olefins, may optionally be recycled back into the alkylation unit 54 via the line 519 as well as the part of unreacted aromatic 517 via line 520.

An aromatic containing stream (e.g. reformate), may also be added to the feed of alkylation unit 54 via line 515.

FIG. 6 shows an oligomerization and alkylation process scheme using a purification section and optionally a recycle of unreacted olefin and aromatic streams.

The untreated refinery stream 610 is fed to a Selective Hydrogenation Unit, SHU 61. The dedienized refinery stream 611 thus obtained is separated in a first splitter 62 into a LCCS 612 (FBP=60° C. to 100° C.) and a M/HCCS 613 (Boiling point cut ranges from 60-170° C. to 100-170° C.).

LCCS 612 is then purified in a purification unit 63 for removal of nitrogen by adsorption on clay sorbent material, optionally further with an adsorbent as previously defined.

The purified stream 614 is fed to an oligomerization unit 64.

The effluents issued from the oligomerization unit 64 are separated in another splitter 65 into a stream 616 of oligomers and unreacted olefins, a part of which may optionally be recycle upstream of oligomerization unit 64 via line 615, and into a stream of (light) middle distillates 617.

Middle distillates 617 are fed to an alkylation unit 66 which is also fed with a (light) gasoline cut 618. Effluents issued from the alkylation unit 66 are separated in a third splitter 67 into a (heavier) gasoline cut 620, a part of which may be recycled back upstream of alkylation unit 66 via line 619, and into a (heavier) middle distillate 621.

FIG. 7 shows a one pot oligomerization and alkylation process scheme using a purification section and optionally a recycle of unreacted olefin stream.

The untreated refinery stream 710 is fed to a Selective Hydrogenation Unit, SHU 71. The dedienized refinery stream 711 thus obtained is separated in a first splitter 72 into a LCCS 712 (FBP=60° C. to 100° C.) and a M/HCCS 713 (Boiling point cut ranges from 60-170° C. to 100-170° C.).

LCCS 712 is then purified in a dedicated purification unit 73 and M/HCCS 713 is purified in another dedicated purification unit 73′, for removal of nitrogen by adsorption on clay sorbent material, optionally further with an adsorbent as previously defined.

The purified streams 714 and 718 issued respectively from purification units 73 and 73′, are fed to a combined oligo-alkylation unit 74.

The effluents issued from the oligo-alkylation unit 74 are separated in a further splitter 75 into a stream 716 of middle distillates, a part of which may optionally be recycled upstream of oligo-alkylation unit 74 via line 715, and into a gasoline cut 717.

FIG. 8 shows an oligomerization and alkylation process scheme of a full olefin containing refinery stream, this process using a purification section and optionally a recycle of unreacted olefin and aromatic streams.

The untreated refinery stream 810 is fed to a Selective Hydrogenation Unit, SHU 81. The dedienized refinery stream 811 thus obtained is separated in a first splitter 82 into a LCCS 812 (FBP=60° C. to 100° C.) and a M/HCCS 813 (Boiling point cut ranges from 60-170° C. to 100-170° C.).

LCCS 812 is then purified in a purification unit 83 on sorbents and M/HCCS 813 is purified in a different purification unit 83′, for removal of nitrogen by adsorption on clay sorbent material, optionally further with an adsorbent as previously defined.

The purified LCCS stream 814 is fed to an oligomerization unit 84. The effluents issued from the oligomerization unit 84 are separated in another splitter 85 into a stream 817 of (light) gasoline cut and a stream 815 of oligomers. The part of unreacted olefins 816 of the stream 817 may be recycled upstream of oligomerization unit 84.

The purified M/HCCS 822 is fed to an alkylation unit 86, preferably with an aromatic containing stream 818. The stream 815 of oligomers issued from splitter 85 is also fed to the alkylation unit 86 The effluents issued from the alkylation unit 86 are separated in a further splitter 87 into a stream 820 of middle distillates and a stream 821 of a (heavier) gasoline cut. The part of unreacted material 819 of the stream 821 may be recycled upstream of alkylation unit 86.

### EXAMPLES

In the following examples, the gasoline cut feedstock is a LCCS cut (Light Catalytic Cracked Stream) corresponding to the low boiling point fraction of a LCN (Light Cracked Naphtha) treated on a Prime-G 1st stage unit (Prime-G is a naphtha selective hydrogenation technology marketed by Axens, which hydrogenates most reactive alkenes, mainly di-olefines, in particular conjugated dienes (e.g. buta-1,3-diene into but-1-ene) and eventually isomerizes n-olefines (end-chain double bond, e.g. n-hex-1-ene) into sec-olefines (internalized double bond, e.g. n-hex-2-ene), so as to get rid of the di-olefins (by selective hydrogenation) and of the low molecular weight sulfur containing molecules by conversion into heavier ones.

Characteristics of the gasoline cut are reported below in Table 1.

TABLE 1
Characteristics of the LCCS cut used in the examples

| | Unit | Value |
|---|---|---|
| Density at 15° C. | g/mL | 0.6518 |
| Sulfur | ppm wt | 24 |
| Total nitrogen | ppm | 12.6 |
| Diene value UOP 326 | g iodine/100 g | 0.11 |
| Bromine Number ASTM D1159 | g bromine/100 g | 140.8 |
| Reid Vapor Pressure ASTM D5191 | kPa | 125.5 |
| Sulfur speciation | | |
| Methyl-ethyl-sulfide | ppm wt | 2 |
| Thiophene | ppm wt | 22 |
| ASTM D86 | | |
| T ° C. at IBP | ° C. | 27.6 |
| T ° C. at 5% vol | ° C. | 31 |
| T ° C. at 50% vol | ° C. | 38 |
| T ° C. at 95% vol | ° C. | 55 |
| T ° C. at FBP | ° C. | 63.6 |
| Chemical species | | |
| C4 | % wt | 6.80 |
| C5 | % wt | 45.44 |
| C6 | % wt | 10.65 |
| Total olefins | % wt | 62.89 |
| iC5 | % wt | 21.35 |

The olefinic conversion is expressed in % wt as:

100×(olefin IN−olefin OUT)/olefin IN

100 mL of ZSM-5 based catalyst diluted with 100 mL of inert material (SiC 0.21 mm) have been loaded in a fixed bed tubular reactor of 18 mm inner diameter. Before testing, catalyst has been activated at 400° C. (60° C./h) under 160 NL/h Nitrogen during 2 hours. Temperature has then been decreased down to 40° C. before starting the testing program.

### Example 1 (Comparative Example)

### Oligomerization without Purification Section

The LCCS cut is directly oligomerized on a ZSM-5 based catalyst (80% wt alumina−20% wt ZSM-5) in the following operating conditions: at 55 barg, with a LHSV (liquid hour space velocity) of 1 h⁻¹, once through, temperature has been increased from 180° C., to 220° C., up to 250° C.

Olefin conversion (% wt) data as a function of TOS when no purification section is used is shown at FIG. 1.

Though the contaminants level is low (24 ppmwt S, 12 ppmwt N), the deactivation of the catalyst is significantly pronounced as represented in the FIG. 1: for instance at 220° C., a loss of 28% wt olefin conversion is observed within 59 hours. Table 2 below gathers the product characteristics obtained for samples withdrawn at different times of stream (TOS).

TABLE 2
Product distribution and density of the effluents recovered at
different TOS when no purification section is used

| | 180° C. | 220° C. | | | 250° C. | |
|---|---|---|---|---|---|---|
| TOS (h) | 16.3 | 31 | 51 | 75 | 116 | 147 |
| IBP-165 | 96.2 | 83.2 | 85.5 | 90.9 | 81 | 94.2 |
| 165-245 | 2.2 | 12.3 | 10.3 | 5.8 | 14.6 | 3.7 |
| 245-350 | 0 | 0 | 0 | 0 | 2.7 | 0 |
| 350-FBP | 0 | 0 | 0 | 0 | 0 | 0 |
| FBP (° C.) | 194 | 266 | 263 | 196 | 272 | 194 |
| Density | 0.6642 | 0.6899 | 0.6847 | 0.675 | 0.6924 | 0.6652 |
| at 15° C. (g/mL) | | | | | | |

This example clearly stresses the need for a process sequence in which catalyst lifetime can be improved.

### Example 2

### Oligomerization with Purification Section

The LCCS cut is first purified on a set of two molecular sieves: 3A followed by 13X, before being oligomerized on the ZSM-5 based catalyst (80% wt alumina−20% wt ZSM-5) used for example 1.

The following operating conditions were chosen: 55 barg, a LHSV (liquid hour space velocity) of 1 h⁻¹, once through, and temperature has been increased from 180° C., to 220° C., up to 250° C.

As observed on FIG. 2, the catalyst deactivation has been largely limited thanks to the use of a purification section.

The product distribution as well as the density of the effluent recovered is reported in table 3 below. The following abbreviations are used:

WABT=Weight Average Boiling Point.

Barg=bar gauge, i.e. absolute pressure minus atmospheric pressure. 1 barg=2 bar absolute (or 2 bara) when referred to a complete vacuum, and when atmospheric pressure is 1 bar.

Analyses of the LCCS purified on molecular sieves have been achieved and reveal that:
The breakthrough of sulfur compounds (mainly thiophenic) is quickly reached (after only 2 days of run, the same sulfur content is obtained at the outlet of the molecular sieves)
Nitrogen containing species are successfully retained on the purification section (the N content is found below 0.5 ppmwt).

The comparison of the results obtained in presence and in absence of a purification section (FIGS. 1&2) clearly underlines the beneficial effect of the use of a purification section prior to acid catalyzed reactions (such as oligomerization, alkylation . . . ). The larger impact of small nitrogen containing molecules compared to aromatic sulfur containing molecules (e.g. thiophene) on catalyst deactivation is also clearly stressed here.

Boiling points are given at ambient pressure, unless otherwise specified.

TABLE 3
Product distribution (%) and density of the effluents recovered
at different TOS when a purification section is used.

| | Pressure in operation | | | | | | |
|---|---|---|---|---|---|---|---|
| | 55 barg | | | | | | |
| TOS (h) | 16 | 31.5 | 55.5 | 79.5 | 103.5 | 127.5 | 151.5 |
| WABT | 182.2 | 182.2 | 202.9 | 202.8 | 202.7 | 198.5 | 199.6 |
| (° C.) | | | | | | | |
| IBP-165 | 76.7 | 76.6 | 65.8 | 66.3 | 66.5 | 69.3 | 69.5 |
| 165-245 | 17 | 17 | 21.3 | 21.3 | 21.1 | 20.5 | 20.5 |
| 245-350 | 3.6 | 3.1 | 10.4 | 10 | 10 | 8.9 | 8.7 |
| 350-FBP | 0 | 0 | 0 | 0 | 0 | 0 | 0 |
| FBP (° C.) | 271 | 269 | 329 | 326 | 326 | 320 | 318 |
| Density at | 0.7036 | 0.7033 | 0.716 | 0.7156 | 0.7153 | 0.713 | 0.7128 |
| 15° C. | | | | | | | |
| (g/mL) | | | | | | | |

| | Pressure in operation | | | | | |
|---|---|---|---|---|---|---|
| | 25 barg | | | | 55 barg | |
| TOS (h) | 175.5 | 223.5 | 247.5 | 271.5 | 295.5 | 343.5 |
| WABT (° C.) | 203.5 | 200.2 | 200.2 | 200.1 | 199.4 | 223.1 |
| IBP-165 | 67.8 | 70.7 | 71.5 | 71.7 | 71.5 | 60 |
| 165-245 | 20.7 | 20.1 | 19.5 | 19.4 | 20.2 | 23 |
| 245-350 | 9.6 | 8.2 | 7.6 | 7.2 | 6.8 | 14.2 |
| 350-FBP | 0 | 0 | 0 | 0 | 0 | 1 |
| FBP (° C.) | 323 | 319 | 317 | 316 | 316 | 360 |
| Density at 15° C. | 0.7141 | 0.7106 | 0.7099 | 0.7093 | 0.7104 | 0.7184 |
| (g/mL) | | | | | | |

In the following examples 3 through 5, nitrogen removal using different adsorbents is shown for a selected feedstock. Said selected feedstock is a second light cycle naphtha (LCN), the properties of which are given in tables 4 to 7, below.

Second LCN Characteristics:

TABLE 4
General physico-chemical properties

| Second LCN | | |
|---|---|---|
| Density | 0.7213 | |
| Total Sulfur content | 196 | ppmw |
| Total Nitrogen content | 17.1 | ppmw |
| Basic Nitrogen content | 13.8 | ppmw |
| Nitrile Nitrogen content | 2.0 | ppmw |
| Water content ASTM D1744 (Karl | 0.015% | wt |
| Fisher) | | |
| Reid Vapor Pressure ASTM D5191 | 52.9 | kPa |

TABLE 5
Sulfur speciation (Total sulfur content = 196 ppm)

| Second LCN Compound | ppm S |
|---|---|
| Thiophene + Butan-2-thiol | 21 |
| Unidentified compounds | 2 |
| 2-methyl-thiophene | 25 |
| 3-methyl-thiophene | 34 |
| Thiacyclopentane | 10 |
| Methyl-thiacyclopentane | 8 |
| 2-ethyl-thiophene | 12 |
| 2,5-dimethyl-thiophene | 4 |
| Other C2 containing thiophenes | 34 |
| C3 containing thiophenes | 21 |
| Over C3 containing thiophenes | 15 |
| Benzothiophene (benzo[b]- and | 5 |
| benzo[c]-thiophene isomers) | |
| Over benzothiophenes | 5 |

TABLE 6
Nitrogen speciation (Total nitrogen content = 17.1 ppm)

| Second LCN Compound | ppm N |
|---|---|
| Acetonitrile | 0.2 |
| Propionitrile | 0.8 |
| Iso-butyronitrile | 0.2 |
| n-butyronitrile | 0.5 |
| Pyrazine like compound | 0.3 |
| Pyridine | 0.8 |
| Valeronitrile | 0.3 |
| Aniline | 3.3 |
| C1-anilines | 3.8 |
| C2-anilines | 2.3 |
| C3-anilines | 1.7 |
| Over C3-anilines | 1.6 |
| Unidentified nitrogen compounds | 1.3 |

TABLE 7
LCN properties: ASTM D86 distillation

| Entry | Result | |
|---|---|---|
| T ° at IBP | 36.5° | C. |
| T ° at 5% vol. | 53° | C. |
| T ° at 10% vol. | 57.3° | C. |
| T ° at 20% vol. | 64.1° | C. |
| T ° at 30% vol. | 71.5° | C. |
| T ° at 40% vol. | 79.8° | C. |
| T ° at 50% vol. | 89° | C. |
| T ° at 60% vol. | 98.7° | C. |
| T ° at 70% vol. | 109° | C. |
| T ° at 80% vol. | 122° | C. |
| T ° at 90% vol. | 141.4° | C. |
| T ° at 95% vol. | 166° | C. |
| T ° at FBP | 179.5° | C. |
| % recovered at 70° C. | 28% | vol. |
| % recovered at 100° C. | 61.4% | vol. |
| % recovered at 150° C. | 92.1% | vol. |
| Recovered % vol. | 97% | vol. |
| Residue % vol. | 1.3% | vol. |
| Loss % vol. | 1.7% | vol. |

Chemical composition and repartition of second LCN. PIONA speciation (Paraffins, Iso-paraffins, Olefins, Naphthenes, Aromatics) is shown in table 8, below:

TABLE 8
Normalized weight percent results

| | | | n- | Cycl. | | | | |
|---|---|---|---|---|---|---|---|---|
| C-nr | Naph | i-Par | par | Ol. | i-olef. | n-olef. | Arom. | Total |
| 4 | | 0.18 | 0.36 | | 0.24 | 1.82 | | 2.6 |
| 5 | 0.24 | 6.84 | 1.09 | 0.55 | 5.62 | 5.4 | | 19.74 |
| 6 | 2.69 | 7.49 | 0.88 | 2.46 | 6.35 | 3.82 | 0.89 | 24.58 |
| 7 | 4.37 | 5.81 | 0.6 | 3.09 | 4.92 | 2.16 | 3.94 | 24.89 |
| 8 | 3.07 | 3.75 | 0.34 | 1.33 | 2.81 | 0.95 | 4.8 | 17.05 |
| 9 | 1.2 | 1.79 | | 0.3 | 1.3 | | 3 | 7.59 |
| 10 | 0.32 | 0.71 | | 0.06 | 0.26 | 0.13 | 1.05 | 2.53 |
| 11 | 0.23 | 0.07 | | | 0.08 | 0.16 | | 0.54 |
| 12+ | | 0.31 | | | | | 0.17 | 0.48 |
| Poly | | | | | | | | |
| Total | 12.12 | 26.95 | 3.27 | 7.8 | 21.58 | 14.43 | 13.86 | 100.01 |

(C-nr = number of carbon atoms within considered compounds; Naph = naphthenes; i-par = iso-paraffins; n-par = normal-paraffins, Cycl. Ol. = cyclic olefins, i-olef. = iso-olefins, n-olef. = normal-olefins; Arom. = aromatics).

### Example 3

### Second LCN Pretreatment Using Molecular Sieves

25 cm³ of standard commercial 13X molecular sieves is calcinated at 200° C. overnight. When cooled down to room temperature, 13X is placed into a double-walled tubular reactor (diameter=1.6 cm, length=29 cm, volume=58.3 cm³) with a sintered glass septum.

Second LCN is Introduced at the Inlet of the Reactor.

The outlet line of the reactor is linked with an automatic sampler provided with 50 glass tubes of 150 cm³ capacity, wherein the out coming treated feed is collected at regular times. The adsorption test is carried out in up-flow mode at a LHSV=2 h⁻¹ (feed rate=50 cm³/h) for several hours at 25° C. and atmospheric pressure. (LHSV=Liquid Hourly Space Velocity).

Nitrogen compounds breakthrough using 13X molecular sieves according to example 3, is shown in FIG. 9. As can be seen from said FIG. 9, nitrogen breakthrough starts after about 20 h, and after 70 h in run, almost 70% of total nitrogen content of the feed is not retained.

### Example 4

### Second LCN Pretreatment Using Alumina Alone or in Combination with Molecular Sieves

The procedure described in Example 3 was repeated with activated alumina SAS-451 (Axens) as well as a combination of 13X and SAS-451, as recommended by the producer. Activated alumina was tested both calcinated and not calcinated, which shown equivalent results. For sake of clarity, only the N-breakthrough results obtained from the non-calcinated alumina are presented in FIG. 10. N-breakthrough is shown for the two systems. 13X trend alone is also reported for a direct evaluation of performances.

As can be seen from FIG. 10, SAS-451 has no nitrogen adsorption capabilities by itself. When coupled with the molecular sieves, retention is pretty improved but no impacts clearly appear in nitrogen breakthrough.

### Example 5

The procedure described in Example 3 was repeated with Filtrol clay F24 (BASF) while no calcination was done before run (residual acidity=11 mg KOH/g clay).

FIG. 11 shows performances in nitrogen retention of F24 system and 13X graph is shown as reference.

When using F24 sorbent, nitrogen concentration keeps constant at 1.8-1.9 ppmwt during a period of 84 hours. Nitrogen concentration is still in the range of 2-2.2 ppmwt after 102 h of run, and then rises slightly over 10 ppmwt after 200 h.

PIONA analyses have been performed during the test in order to verify that olefins were still present and in the same concentration and proportion, and that secondary reactions such as isomerization or oligomerization did not occur.

Nitrogen repartition was measured at the outlet of the F24 section after 48 h of run. Results are given in table 9, below.

TABLE 9

| | | Out of F24 section |
|---|---|---|
| | Feedstock | after 48 h run |
| | (total Nitrogen = | (total Nitrogen = |
| Chemical species | 17.1 ppmw) | 1.7 ppmw) |
| Acetonitrile | 0.2 | 0.4 |
| Propionitrile | 0.8 | 0.4 |
| Iso-Butyronitrile | 0.2 | 0.1 |
| n-Butyronitrile | 0.5 | 0.3 |
| Pyrazine (?) | 0.3 | 0.2 |
| Pyridine | 0.8 | 0.2 |
| Valeronitrile | 0.3 | — |
| Aniline | 3.3 | — |
| C1-Anilines | 3.8 | — |
| C2-Anilines | 2.3 | — |
| C3-Anilines | 1.7 | — |
| C3+-Anilines | 1.6 | — |
| Unidentified nitrogen | 1.3 | 0.1 |
| containing compounds | | |

Nitriles such as acetonitrile or propionitrile are scarcely retained within F24 clay, while other nitrogen containing species such as anilines are removed to a large extent.

### Example 6

The procedure described in Example 3 was repeated with Filtrol clay F25 (BASF). As in the case of Filtrol F24 clay no calcination was done before run (residual acidity=11 mg KOH/g clay).

The two clays were then tested under LHSV=3 h⁻¹ condition as well. The comparisons are shown in FIG. 12 hereinbelow.

During the test at LHSV=2 h⁻¹ the systems show a constant nitrogen retention: for F24 clay total nitrogen is set around 1.8 ppmwt until 84 h of run while F25 clay exhibits a lower retention capacity (2.5 ppmwt all along the test).

Test under LSHV=3 h⁻¹ condition allows to further differentiate the performances of the two clays: for F25 N-compounds retention is constant until about 20 h at 2.5 ppmw than it quickly rises up; F24 demonstrates a better stability on time, showing the same behavior than in the case at LHSV=2 h⁻¹ at least until 50 h of run.

### Example 7

The procedure described in Example 3 was repeated with the combination of molecular sieve 13X and Filtrol F24 clay. Prior to performing the adsorption test the clay was not calcined.

In FIG. 13 hereinbelow the performances of the two combinations 13X/F24 (13X at the bottom of the reactor) and F24/13X (F24 at the bottom of the reactor) are shown. Single 13X and F24 clay are shown as well for comparison.

As already demonstrated in previous Example 5, F24 system is able to adsorb basic nitrogen compounds but still 1.8 ppmwt of nitriles break through from the beginning of the run; in Example 3 for 13X molecular sieve the nitrogen breakthrough occurs faster than the case of F24, but residual nitriles content is only 0.5 ppmwt until 20 h of run. Then, concerning the combinations of the two systems, 13X molecular sieve allows to remove the residual nitriles not retained by the clay; anyway the nitriles breakthrough is not improved as a whole.

1. A method for preparing a nitrogen-depleted hydrocarbon feedstock having (i) an initial boiling point comprised between 0° C. and +180° C. and a final boiling point comprised between 30° C. and 250° C., and (ii) an olefin content higher than 5 weight %, by contacting a hydrocarbon feedstock starting material with a clay sorbent material in a reaction vessel, wherein the nitrogen-depleted hydrocarbon feedstock has a total nitrogen/nitrile ratio (ppm/ppm) comprised between 1 and 5.

2. The method according to claim 1, wherein said clay sorbent material is selected among kaolinite, montmorillonite-smectite, illite and chlorite.

3. The method according to claim 1, wherein the clay sorbent material is a hydrated acid treated smectite clay selected among montmorillonite, bentonite, vermiculite, hectorite, saponite, and beidillinite.

4. The method according to claim 3, wherein the clay sorbent material is a magnesium-substituted hydrogen montmorillonite.

5. The method according to claim 1, wherein residual acidity in said clay sorbent material is greater than 3 mg KOH per gram of said clay sorbent material.

6. The method according to claim 4, wherein said clay sorbent material of magnesium-substituted hydrogen montmorillonite is selected among Filtrol F24, F124, F224, F25, F71 and Tonsil CO—N clays.

7. The method according to claim 1, wherein the hydrocarbon feedstock starting material, having a total nitrogen/nitrile ratio (ppm/ppm) higher than 1, is contacted with said clay sorbent material within the reaction vessel, at a temperature comprised between the nitrogen-depleted hydrocarbon feedstock freezing point and final boiling point, with a liquid hourly space velocity (LHSV) lower than 4 h⁻¹, and a pressure comprised between 1 bar and 55 bar.

8. The method according to claim 7, wherein the hydrocarbon feedstock starting material is contacted with said clay sorbent material at a temperature comprised between 0° C. and 100° C., with a LHSV between 3 h⁻¹ and 0.5 h⁻¹, and a pressure comprised between atmospheric pressure and 5 bar.

9. The method according to claim 1, wherein the nitrogen-depleted hydrocarbon feedstock is further contacted with an adsorbent, wherein said adsorbent includes one or more of molecular sieves, acidic ion-exchange resins, activated alumina, spent FCC catalysts, MOF (Metal-Organic Framework), ASA, NiMo, and catalysts guard beds, or mixture thereof.

10. The method according to claim 9, wherein the adsorbent is selected among, or is a combination of one or more of 13X, 3A molecular sieves, ASA, NiMo, and MOF.

11. A nitrogen-depleted hydrocarbon feedstock exhibiting a total nitrogen/nitrile ratio (ppm/ppm) comprised between 1 and 5, obtained by the method according to claim 1.

12. An olefin oligomerization and/or alkylation process, comprising using the hydrocarbon feedstock according to claim 11.

13. A process for olefin oligomerization and/or alkylation of a nitrogen depleted hydrocarbon feedstock containing olefins, comprising:
(i) selective hydrogenation of a hydrocarbon feedstock starting material containing olefins,
(ii) treatment of the resulting hydrocarbon feedstock on a clay sorbent material, to obtain at least one nitrogen depleted hydrocarbon feedstock;
(iii-a) oligomerization and/or (iii-b) alkylation of said nitrogen-depleted feedstock, for at least one middle distillate production.

14. A nitrogen-depleted hydrocarbon feedstock having (i) initial boiling point comprised between 0° C. and +180° C. and a final boiling point comprised between 30° C. and 250° C., (ii) an olefin content higher than 5 weight %, (iii) a total nitrogen/nitrile ratio (ppm/ppm) comprised between 1 and 5, prepared by using a clay sorbent material.
